(19) United States
(12) Patent Application Publication
(10) Pub. No.: US 2024/0057881 A1
(43) Pub. Date: Feb. 22, 2024

(54) System and Method for PPG Using Wideband Accelerometer with a Wearable Device

(71) Applicant: Google LLC, Mountain View, CA (US)

(72) Inventors: Sherk Chung, Mountain View, CA (US); Ian Atkinson, Mountain View, CA (US); Saket Patkar, Mountain View, CA (US)

(73) Assignee: Google LLC, Mountain View, CA (US)

(21) Appl. No.: 18/255,572

(22) PCT Filed: Dec. 22, 2020
(86) PCT No.: PCT/US2020/066635

§ 371 Date: Jun. 1, 2023

(51) Int. Cl. A61B 5/024; A61B 5/00

(52) U.S. Cl. CPC A61B 5/02427; A61B 5/721; A61B 5/681; A61B 2562/0219; A61B 5/6817; A61B 5/0002

(57) ABSTRACT

An apparatus includes a processing device coupled to a memory storing instructions. The instructions may cause the processing device to receive photoplethysmography (PPG) data derived from signals associated with a PPG sensor oriented proximate a portion of an anatomy of a human subject and associated with a heart rate of the human subject; receive wb-accelerometer data derived from signals associated with a wb-accelerometer sensor oriented to sense the heart rate the human subject; and combine the PPG data and the wb-accelerometer data to generate a heart rate estimate.

### BACKGROUND

Photoplethysmography (PPG) is an optical measurement method which measures changes in tissue volume and requires a light source and a photodetector. A photodetector, typically placed at or close to the surface of skin, detects light which is either transmitted or reflected from vascular tissue to the photodetector. This light corresponds to variations in the volume of blood circulation, which can be used to monitor heart rate. The change in volume caused by a pulse or cardiac cycle can be measured as a peak or trough in the intensity of light. The photodiode measures the light intensity in the tissue and an algorithm can be used to translate the variation in intensity to a computed heart rate. The technique can also be used to measure other aspects related to blood flow, such as oxygen saturation level of the blood.

The accuracy of the computed heart rate is typically proportional to the signal-to-noise ratio (SNR) of the PPG signal. Accuracy can be improved by using a stronger light source, or by reducing noise in the system. For some devices, such as smart watches for example, power constraints limit use of available techniques to improve SNR. Such techniques can include for example increasing power to the LED or increasing the PPG sensor sampling rate.

Further, despite an increase in SNR of the PPG signal at the expense of increasing power, other degradations or sources of error can be introduced which may impact the accuracy of the measured parameters. For example, “motion artifacts” can be introduced into the PPG signal owing to physical motion of a user rather than a change in the heart rate itself. For example, when a person is wearing a wrist band with a PPG sensor, the normal movement of the arm, such as swinging the arm, can introduce artifacts in the PPG signal which can be an order of magnitude larger than the artifacts related to the heart rate. Such motion artifacts degrade the accuracy of common wearable PPG systems, and can furthermore introduce false positive readings where the algorithm produces erroneous results based on motion artifacts.

### SUMMARY

According to aspects of the disclosure, an apparatus is disclosed that comprises a processing device coupled to a memory storing instructions. The instructions may cause the processing device to receive photoplethysmography (PPG) data derived from signals associated with at least one PPG sensor; receive wideband accelerometer data derived from signals associated with at least one wideband accelerometer sensor configured to sense a heart rate of a human subject; and combine the PPG data and the wideband accelerometer data to generate a heart rate estimate. In one example, the apparatus includes at least one of the at least one PPG sensor and the at least one wideband accelerometer sensor.

In one example, the apparatus comprises both the at least one PPG sensor and the at least one wideband accelerometer sensor.

In an example, the instructions may cause the processing device to filter PPG data with general purpose accelerometer data from at least one general purpose accelerometer sensor.

In another example, the apparatus comprises an ear-worn wearable device.

In another example, the apparatus comprises a wrist worn device.

In another example, the apparatus comprises at least one filter that can filter signals outside a frequency band associated with heart rate frequencies from the wideband accelerometer data. The frequency band can include a frequency of at least 0.6 Hz, and may include frequencies between 0.6 to 2 Hz.

In an example, instructions may cause the processing device to normalize the wideband accelerometer data prior to combining the wideband accelerometer data with the PPG data.

In another example, the instructions may cause the processing device to combine using a weighted combination in which the PPG data and the wideband accelerometer data are scaled with a weighting factor and summed.

In another example, the instructions may cause the processing device to combine the PPG data and the wideband accelerometer data using one of simple averaging, weighted averaging, kalman filtering and bayesian networks.

In still another example of this aspect, the instructions may cause the processing device to identify noise artifacts in the PPG data based on the wideband accelerometer data.

In another example, the instructions may cause the processing device to combine one or more samples of wideband accelerometer data with one or more samples of PPG data by using the wide band accelerometer data as an additive source with the PPG data.

In yet another example, the wideband accelerometer data can be used to compute a confidence metric for a PPG heart rate estimate derived from the PPG data.

In yet another example, the processing device receives the PPG data and the wideband accelerometer data over a wireless link.

According to another aspect of the disclosure, the apparatus may comprise the at least one PPG sensor and/or the at least one wideband accelerometer sensor, such as, at least one PPG sensor and/or at least one wideband accelerometer sensor configured and arranged to sense the heart rate of a human subject. The apparatus can also comprise a housing, wherein both the PPG sensor and the wideband accelerometer sensor are arranged within the housing. In some examples, the PPG sensor can be configured to be oriented proximate a portion of an anatomy of a human subject.

In some examples, the wideband accelerometer sensor may be arranged within the apparatus and configured to be oriented proximate an ear canal of the human subject. For example, the apparatus can comprise an ear-worn wearable device, wherein, for example, the wideband accelerometer sensor is a part of the ear-worn wearable device. For example, the housing mentioned above may be part of the ear worn wearable device, and the PPG sensor and the wideband accelerometer sensor may be arranged within the housing of the ear-worn device. The ear-worn wearable device of the apparatus may be the ear-worn wearable device according to another aspect of the invention described below.

As an alternative or in addition, the apparatus according to the disclosure may also comprise a wrist worn device or another wearable device, wherein, for example, at least one wideband accelerometer sensor is part of the wrist worn device or the other wearable device.

In some examples, the processing device may receive the PPG data and/or the wideband accelerometer data over a wireless link. However, it is of course also possible that the PPG data and/or the wideband accelerometer data are received by the processing device via a wired link. For example, the processing device may be arranged in a housing together with the PPG sensor and/or the wideband accelerometer sensor and, for example, connected to the sensor(s) via a wired link. The housing may be part of an ear-worn wearable device as already set forth above.

In other examples, the apparatus may comprise at least one filter to filter from the wideband accelerometer data, signals outside a frequency band associated with heart rate frequencies. For example, the at least one filter is implemented by the instructions stored in the memory coupled to the processing device. Further, the instructions may cause the processing device to filter the PPG data with general purpose accelerometer data from a general purpose accelerometer. For this purpose, the apparatus according to aspects of the disclosure may further comprise the general purpose accelerometer.

According to another aspect of the disclosure, an apparatus includes a processing device coupled to a memory storing instructions. The instructions may cause the processing device to compute a first heart rate estimate from PPG data associated with at least one PPG sensor; compute a second heart rate estimate from wideband accelerometer data associated with at least one wideband accelerometer sensor configured to sense a heart rate of a human subject; and combine the first and second heart rate estimates to generate a third heart rate estimate.

In one example, the apparatus may be or include any of the features of the apparatus discussed above.

In one example, the at least one wideband accelerometer sensor is designed to have a frequency response bandwidth of at least 2 kHz.

In another example, instructions may cause the processing device to filter PPG data with general purpose accelerometer data derived from the at least one general purpose accelerometer sensor, and wherein the at least one general purpose accelerometer sensor detects frequency bandwidths less than 2 kHz.

According to another aspect of the disclosure, an apparatus includes a processing device coupled to a memory storing instructions. The instructions cause the processing device to compute a first heart rate estimate from PPG data associated with at least one PPG sensor; compute a second heart rate estimate from wideband accelerometer data associated with at least one wideband accelerometer sensor configured to sense a heart rate of a human subject; and compare the first heart rate estimate to the second heart rate estimate to determine a final heart rate estimate. When a difference between the first and second heart rate estimates is within a threshold, the first heart rate estimate can be used to compute the final heart rate estimate.

In one example, the apparatus may be or include any of the features of the apparatus discussed above.

In one example, when the difference falls outside of the threshold, the instructions may cause the processing device to discard the first heart rate estimate.

According to another aspect of the disclosure, an ear-worn wearable device is disclosed. The ear-worn device comprising at least one a photoplethysmography (PPG) sensor may be configured to be oriented proximate a portion of an anatomy of a human subject; and at least one wideband accelerometer sensor may be configured to sense a heart rate of the human subject. In one example, the ear-worn wearable device may be for an apparatus as discussed above.

In one example, the ear-worn wearable device comprises a housing. At least one of the at least one PPG sensor and the at least one wideband accelerometer sensor is arranged within the housing.

In another example, both the at least one PPG sensor and the at least one wideband accelerometer system are arranged in the housing.

In another example, the housing comprises an ear canal portion that is configured to be arranged proximate or at least partially within an ear canal of the human subject. In one example, the wideband accelerometer sensor is arranged at least partially within the ear canal portion.

In another example, a processing device may be coupled to a memory storing instructions. The instructions may cause the processing device to receive PPG data derived from signals associated with the at least one PPG sensor and associated with a heart rate of the human subject; receive wideband accelerometer data derived from signals associated with at least one wideband accelerometer sensor; and combine the PPG data and the accelerometer data to generate a heart rate estimate.

In one example, the processing device is arranged in a housing.

In another example, the at least one wideband accelerometer sensor is configured to sense vibration having a vibrational frequency above 100 Hz.

In still another example, the ear-worn wearable device comprises a feed forward microphone.

According to another aspect of the disclosure, an ear-worn wearable device includes at least one a photoplethysmography (PPG) sensor configured to be oriented proximate a portion of an anatomy of a human subject; and at least one wideband accelerometer sensor configured to sense the heart rate of the human subject.

In one example, the ear-worn wearable device may comprise a housing, wherein both the PPG sensor and the wideband accelerometer sensor are arranged in the housing. The PPG sensor can include a light source (e.g., an LED) and a light detector (e.g., a semiconductor photodetector).

For example, the housing can comprise an ear canal portion (e.g., a tip) that is configured to be arranged proximate or at least partially within an ear canal of the human subject. The ear-worn wearable may be attached to the wearer's ear by introducing at least a part of the ear canal portion into the ear canal. The wideband accelerometer sensor may be arranged at least partially within the ear canal portion.

In some examples, the ear-worn wearable device may comprise a processing device coupled to a memory storing instructions. The instructions may cause the processing device to receive PPG data derived from signals associated with the PPG sensor and associated with a heart rate of the human subject; receive wideband accelerometer data derived from signals associated with the wideband accelerometer sensor; and combine the PPG data and the wideband accelerometer data to generate a heart rate estimate. In particular, the processing device and the instructions may be configured as described above in conjunction with the apparatus according to aspects of the disclosure. The processing device may be a programmable unit, such as a CPU or a component of a CPU.

In some examples, the processing device may be arranged in the housing mentioned above such that the PPG sensor, the wideband accelerometer sensor and the processing device (e.g., including the memory) are arranged together within the housing. The PPG sensor, the wideband accelerometer sensor and the processing device may form a module arranged in the housing. For example, the PPG sensor, the wideband accelerometer sensor and the processing device are arranged in a common housing and/or on a common substrate (e.g., a circuit board). The module may comprise a power source for supplying power to the PPG sensor and/or the wideband accelerometer sensor. Moreover, the module may comprise other sensors in addition to the PPG sensor and the wideband accelerometer sensor (e.g., a general purpose accelerometer sensor) and/or a communication interface. In some examples, the wideband accelerometer sensor may configured to sense vibration having a vibrational frequency above 2 kHz, e.g. in the range between 2 kHz and 20 kHz.

Furthermore, the ear-worn wearable device may comprise a feed forward microphone. For example, the feed forward microphone is also arranged in the housing mentioned above.

According to another aspect of the disclosure, a computer-implemented method of determining a heart rate estimate of a human subject is disclosed. The method includes receiving photoplethysmography (PPG) data derived from signals associated with at least one PPG sensor; receiving, from at least one wideband accelerometer sensor, wideband accelerometer data derived from signals associated with a heart rate of the human subject; and determining, at a processing device, the heart rate estimate of the human subject by combining the wideband accelerometer data and the PPG data. In one example, the any one of the apparatus or ear-worn device can be used with the disclosed method.

In one example, the at least one PPG sensor and the at least one wideband accelerometer sensor are housed in a wearable device.

In another example, the wearable device is an ear-worn wearable device.

In still another example, the wearable device comprises a wrist worn device.

In yet another example, the method further includes the processing filtering wideband accelerometer data that is outside a frequency band associated with heart rate frequencies. The frequency band can include a frequency of at least 0.6 Hz, and including frequencies between 0.6 to 2 Hz.

In another example, combining comprises summing a weighted combination of the wideband accelerometer data and the PPG data. The PPG data and the wideband accelerometer data are scaled with a weighting factor.

In still another example, the computer-implemented method of claim any of claims 33 to 34, wherein combining, by the processing device, comprises combining by one of simple averaging, weighted averaging, kalman filtering and using bayesian networks.

In another example, prior to combining, the processing device can subtract noise artifacts from the PPG data based on the wideband accelerometer data.

In another example, the processing device normalizes wideband accelerometer data prior to the combining the wideband accelerometer data with the PPG data.

In still another example, the processing device filters PPG data with general purpose accelerometer data from a general purpose accelerometer sensor.

According to another aspect of the disclosure, a computer-implemented method of determining a final heart rate estimate of a user is disclosed. The method comprises computing a first heart rate estimate from PPG data associated with at least one PPG sensor; computing a second heart rate estimate from wideband accelerometer data associated with at least one wideband accelerometer sensor configured to sense a heart rate of a human subject; and combining the first heart rate estimate and the second heart rate estimate to determine the final heart rate estimate.

In another example, the method can be used with any apparatus or ear worn device described above.

In still another example, the method comprises displaying an alert based on the final heart rate estimate on a display interface. The alert can indicate that the heart rate estimate is above a threshold related to a health state of the subject.

According to another aspect of the disclosure, a computer-implemented method of determining a final heart rate estimate of a user is disclosed. The method includes computing a first heart rate estimate from PPG data associated with at least one PPG sensor; computing a second heart rate estimate from wideband accelerometer data associated with at least one wideband accelerometer sensor; and comparing the first heart rate estimate to the second heart rate estimate to determine the final heart rate estimate, wherein when a difference between the first and second heart rate estimates is within a threshold, the first heart rate estimate is used to compute the final heart rate estimate. In one example, the method can be used with any apparatus or ear worn device described above.

In one example, the first heart rate estimate is disregarded when the difference between the first heart rate estimate and the second hear rate estimate is outside of the threshold.

According to another aspect of the disclosure, a non-transitory computer-readable medium storing software comprising instructions executable by one or more computers is disclosed. Upon execution, the instructions may cause the one or more computers to perform operations including receiving photoplethysmography (PPG) data derived from signals associated with at least one PPG sensor and associated with heart rate of a human subject; receiving wideband accelerometer data derived from signals associated with at least one wideband accelerometer sensor; and determining, at a processing device, a heart rate estimate of the human subject by combining the wideband accelerometer data and the PPG data.

In one example, the processing devices filters one or more of the wideband accelerometer data that is outside a frequency band associated with heart rate frequencies. In one example, the frequency band comprises frequencies at least 0.6 Hz, including frequencies between 0.6 to 2 Hz.

In still another example, the processing device normalizes the wideband accelerometer data prior to the combining the wideband accelerometer data with the PPG data.

In still another example, a weighted combination of the wideband accelerometer data and the PPG data is summed. First samples of the PPG data and second samples of the wideband accelerometer data are scaled with a weighting factor.

In one example, the processing device includes combining by one of simple averaging, weighted averaging, kalman filtering and using bayesian networks.

In another example, prior to combining, the processing device subtracts noise artifacts from the PPG data based on general purpose accelerometer data.

According to another aspect of the disclosure, a non-transitory computer-readable medium storing software comprising instructions executable by one or more computers is disclosed. Upon execution, the instructions may cause the one or more computers to perform operations including computing a first heart rate estimate from PPG data received from signals associated with at least one PPG sensor; computing a second heart rate estimate from data derived from signals associated with at least one wideband accelerometer sensor configured to sense the heart rate; and combining the first and second heart rate estimates to determine a third heart rate estimate.

According to another aspect of the disclosure, a non-transitory computer-readable medium storing software comprising instructions executable by one or more computers is disclosed. Upon execution, the instructions may cause the one or more computers to perform operations including computing a first heart rate estimate from PPG data associated with at least one PPG sensor; computing a second heart rate estimate from wideband accelerometer data received by at least one wideband accelerometer sensor associated with heart rate of a human subject; and comparing the first heart rate estimate to the second heart rate estimate to determine a final heart rate estimate. When a difference between the first and second heart rate estimates is within a threshold, the first heart rate estimate is used to compute the final heart rate estimate.

### BRIEF DESCRIPTION OF THE DRAWINGS

The accompanying drawings are not intended to be drawn to scale. Like reference numbers and designations in the various drawings indicate like elements. For purposes of clarity, not every component may be labeled in every drawing. In the drawings:

FIG. 1 is a schematic view of a PPG module according to aspects of this disclosure.

FIG. 2 is a schematic view of an example portion of the PPG module of FIG. 1 showing additional details according to aspects of this disclosure.

FIG. 3 is a schematic drawing of an example wearable user device according to aspects of this disclosure.

FIG. 4 is a schematic drawing of an example system of user devices according to aspects of this disclosure.

FIG. 5 is a schematic drawing of an example system of user devices according to aspects of this disclosure.

FIG. 6A is a diagram of an example display on a user interface according to aspects of this disclosure.

FIG. 6B is a diagram of an example display on a user interface according to aspects of this disclosure.

FIG. 7 is a schematic diagram of communication between devices according to aspects of this disclosure.

FIG. 8 is a schematic diagram of communication between devices according to aspects of this disclosure.

FIG. 9 is a schematic diagram of communication between devices according to aspects of this disclosure.

FIG. 10A is an example display of PPG data provided by a PPG sensor in a PPG module and associated with heart rate.

FIG. 10B is an example display of wb-accelerometer data provided by a wb-accelerometer sensor in a PPG module and associated with heart rate.

FIG. 10C is a flowchart of an example method according to aspects of this disclosure.

FIG. 11 is a flowchart of an example method according to aspects of this disclosure.

FIG. 12 is a flowchart of an example method according to aspects of this disclosure.

### DETAILED DESCRIPTION

Generally and as non-limiting examples, as used in this disclosure, a “PPG sensor” refers to a circuit that includes a photodiode or other sensor which is capable of measuring light. In some examples, the light for the PPG sensor will arrive from an LED or other light source. “PPG data” can generally refer to the readings from a PPG photodiode. A “PPG algorithm” can generally refer to an algorithm that translates or uses PPG data to generate an estimated heart rate. As one non-limiting example, a “PPG system” can generally refer to a combination of a PPG sensor, a CPU or other computing device which can include memory, and a PPG algorithm which can read PPG data and generate an estimated heart rate. “Wb-accelerometer data” can generally refer to readings from a wideband accelerometer sensor.

### Overview

The disclosed technology in one aspect may comprise methods, systems, and apparatuses which can be used to improve the accuracy of PPG-based and final heart rate estimates by using heart rate data derived from at least one wb-accelerometer sensor and PPG data derived from at least one photodiode or PPG sensor to reduce the error in the PPG-based heart rate estimate. As explained below, in some examples, the apparatus may comprise or may be a wearable user device which contains a PPG module that includes PPG technology, and at least one wb-accelerometer sensor. PPG technology has an inherent error due to numerous possible factors including motion artifacts. The comparison or combination of data from at least one PPG sensor and at least one wb-accelerometer sensor, in either raw form or used to calculate initial PPG heart rate estimates and wb-accelerometer heart rate estimates, can improve the accuracy of the final heart rate estimate as compared to an estimate based on PPG data alone.

### Example Systems

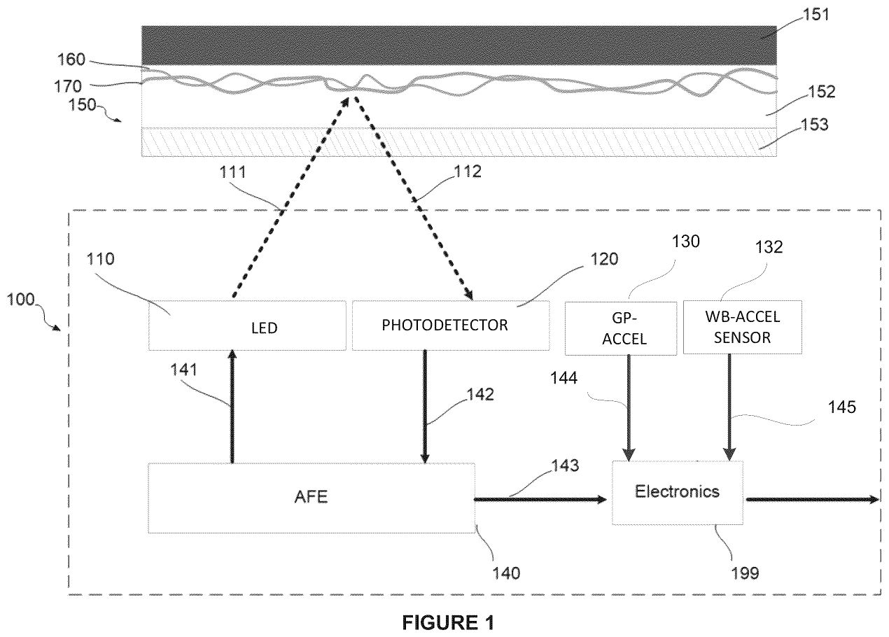

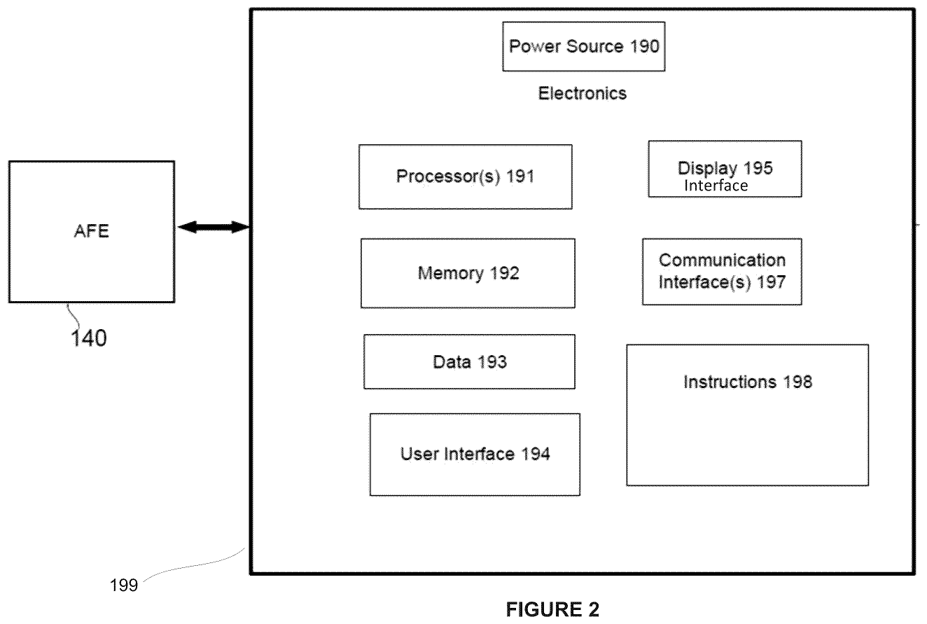

FIG. 1 illustrates an example PPG module 100, which can be used to perform PPG. Module 100 can comprise a light source, such as light source 110, one or more light sensors capable of detecting light, such as photodetector 120, general purpose accelerometer 130, wideband accelerometer 132, analog circuitry to drive the light source or obtain readings from the light sensor, such as analog front end (“AFE”) 140, and electronics 199. In some examples, electronics 199 may include some or all of the features of electronics 199 described below with reference to FIG. 2. In other examples, features, operations, or components of AFE 140 and electronics 199 may be exchanged or combined in various permutations. For instance, electronics 199 is shown as part of the PPG module 100, but in other examples, electronics 199 may exist outside of PPG module 100. Furthermore, electronics 199 can be a processing device, such as a central processing unit.

Rays 111 and 112 are light rays, with the arrow indicating the direction in which the light travels. The light can be incident on a dermis, such as skin 150 with a hypodermis layer 151, a dermis layer 152, and an epidermis layer 153 which may contain vein 160 and artery 170. Although skin 150 is shown, it is possible that the device is applied to other parts of a human body, such as for example, a nail or soft tissue.

Light generated from light source 110, such as ray 111, can be emitted from light source 110 to skin 150. Some of the light emitted from the light source penetrates the skin and is reflected back to photodetector 120, such as ray 112. The reflected light is used to compute an estimated heart rate. Light that reflects off or is transmitted back from these layers is useful for the purpose of PPG.

Variations in the light received by the photodetector can be used to determine various aspects of a cardiovascular system, such as heart rate, pulse, oxygen saturation in the blood, or other health-related information. In some examples, a wave form can be derived from the continuous or near-continuous monitoring of light received by photodetector 120. Light source 110 and photodetector 120 can be connected with AFE 140 to control the emission of light, which can be connected with electronics 199 and to monitor and analyze the light received from skin 150.

Light source 110 can include, but is not limited to a light-emitting diode (LED). An LED is a semiconductor light source which emits light responsive to electricity flowing through it. Electrons in the semiconductor recombine with electron holes, releasing energy in the form of photons. LEDs can be engineered or chosen to emit light at a particular wavelength or range of wavelengths. In other examples, light source 110 can be made of any commercially available source of light, such as lasers, specially designed semiconductors, incandescent light, electrodeless lamps, or halogen lamps. In other examples, light source 110 can further be made of one or more light sources configured to generate light of different wavelengths, such as an LED configured to generate red light which is close to a wavelength of 660 nm, or an LED configured to generated green light which is close to a wavelength of 530 nm. These different light sources may be chosen to measure different aspects of a cardiovascular system when performing PPG. For example, green light may provide information regarding a heartbeat while red light may provide information about blood oxygen saturation, due to the relative absorption and reflection of these colors within the cardiovascular system.

A photodetector, such as photodetector 120, can be a semiconductor device that converts light into an electrical current. The photodetector can generate a current which is proportional to the number of photons hitting a surface of the photodetector. As electricity is generated when photons are absorbed in the photodetector, the photodetector can act as a sensor for light. The photodetector can be any device which is capable of sensing intensities and/or wavelengths of light. Photodetector 120 can be a photodiode or a photosensor. In some examples, photodetector 120 can be chosen to be more sensitive to specific wavelengths of light. In some examples, photodetector 120 can be chosen or configured to be more sensitive or only sensitive to green light while another photodetector can be configured to be more sensitive or only sensitive to red light. Photodetector 120 can also be made of an array of photodetectors. Additional circuitry, calibration, or electronics can be incorporated into the photodetectors, AFE 140, or electronics 199 to ensure a better signal to noise ratio and reduce the effect of ambient light.

In some examples, readings from photodetector 120 can be converted to digital samples at AFE 140. AFE 140 can contain an LED driver and an analog-to-digital converter (ADC). An ADC converts an analog signal into a digital signal. An LED driver can “drive” or control light source 110. AFE 140 can be used to drive light source 110 through a drive signal 141. AFE 140 can also receive an analog signal 142 from photodetector 120. In some examples, AFE 140 can be part of electronics 199, or components of electronics 199, described in more detail below, can be included in AFE 140. AFE 140 can generate information from the analog signal received from photodetector 120, and transfer this PPG data to electronics 199. LED 110 and the detector 120 are part of a PPG sensor. The PPG sensor may also include the AFE 140.

In some examples, PPG data is forwarded to a CPU or processor within electronics 199 via PPG data signal 143, where a PPG algorithm can use information from the PPG data signal 143 to generate a heart rate estimate. PPG data signal 143 can be a digital or analog signal, and may be processed in the time domain or frequency domain. Peak detection techniques, which can use either a time domain or a frequency domain algorithm, can be used to provide heart rate estimates from PPG data alone, but the presence of motion artifacts (MA) can make accurate peak detection challenging. Motion artifacts can occur when a user is not relatively still, causing motion in a portion of the body to change the reflected light being received by photodetector 120. For example, a MA generated when a user is swinging his or her arm can trick PPG algorithms processing data from a PPG sensor worn on the arm into locking onto an incorrect peak or mask the true peak associated with the heart rate of the user.

Data from general purpose accelerometer (“gp-accelerometer”) 130 can be used to filter MA from PPG data to obtain more accurate results. Gp-accelerometer 130 can be any electromechanical 13 device which is configured to measure acceleration responsive to acceleration forces. Gp-accelerometer 130 can generate vectors reflecting acceleration in one or more independent dimensions. Gp-accelerometers have a narrow capture bandwidth. In many examples, a gp-accelerometer captures bandwidth below 2 kHz. GP-accelerometers can provide usable sensitivity for data captured within this narrow range of bandwidths, which allows them to capture forces due to translation and the like. In order to identify peaks created by MA, PPG modules are typically accompanied by a gp-accelerometer. Data 144 from a gp-accelerometer can be used in conjunction with data from or derived from photodetector 120 in a PPG algorithm in a time-domain adaptive filter to cancel out noise generated by motion. In some examples, gp-accelerometer data 144 can be processed with a Fourier transform to identify and filter MA peaks in the frequency domain of the PPG data. Despite these techniques, the cancellation of a motion artifact is difficult and has significant effects on the accuracy. It is also known in the art that cancellation of MA is difficult, which results in lower-than-desired accuracy of the heart rate estimates when the user is in motion, particularly for wrist-worn devices like medical bands and smartwatches where a user is more likely to move his or her arm and the amount of blood being transmitted through the veins and arteries is subject to a greater amount of change based on this motion.

According to aspects of the disclosure, to obtain a more accurate PPG-based and final heart rate estimate that accounts for MA, the PPG algorithm can use data provided by a wideband accelerometer (“wb-accelerometer”) or wb-accelerometer sensor 132 in the PPG module to account for MA. Unlike the gp-accelerometer, a wb-accelerometer sensor 132 can provide usable sensitivity within a much wider range of frequencies, so that the wb-accelerometer can measure vibrations at higher frequencies than a gp-accelerometer. For example, a wb-accelerometer sensor 132 can be used to support higher bandwidths, such as 2 kHz or greater. In some examples, the wb-accelerometer can support a bandwidth in the 2-20 kHz range. This category of accelerometers can capture higher frequency movement and readily sense vibrations. Wb-accelerometer sensor 132 can be any accelerometer device that can detect accelerometer changes related to heart rate, such as pulses caused by heart beats or blood flow. Wb-accelerometer sensor 132 may be configured to measure and analyze the vibrations generated by the pulse of a user or blood pulsing through blood veins or arteries.

According to an aspect of the disclosure, wb-accelerometer data can be forwarded to a CPU or processor within electronics 199 via wb-accelerometer data signal 145, where the PPG algorithm can use information from the wb-accelerometer data signal 145 to generate a final heart rate estimate. Wb-accelerometer data signal 145 may be a digital signal. It is to be appreciated that the wb-accelerometer data can be used in place of the gp-accelerometer data in the PPG algorithm, or it can be used in addition to the gp-accelerometer data.

As noted above, the PPG algorithm can accept wb-accelerometer data and combine it with PPG data to achieve a final heart rate estimate (“final HR estimate”). Utilizing the combination of input data-wb-accelerometer data and PPG data can improve accuracy of the final HR estimate.

Alternatively, the PPG algorithm can accept independently-computed wb-accelerometer and PPG heart rate estimates (computed from PPG data and wb-accelerometer data) that are combined together. For example, wb-accelerometer data alone can first be used to independently determine a first wb-accelerometer heart rate estimate. A second heart rate estimate can be determined based on PPG data alone (or filtered PPG data, such as filtered with general-accelerometer data 144). The first wb-accelerometer heart rate estimate and the second PPG heart rate estimate can then be combined in the PPG algorithm to obtain a PPG based and final HR estimate. Utilizing the combination of output data wb-accelerometer heart rate estimate and PPG heart rate estimate can also improve accuracy of the PPG heart rate estimate.

Finally, a PPG algorithm can compare PPG heart rate estimates with wb-accelerometer heart rate estimates to aid in the identification of the MA in the PPG signal. Peak detection techniques, which can use either a time domain or a frequency domain algorithm, can be used to provide heart rate estimates from PPG data and wb-accelerometer data. An algorithm can then compare PPG peaks, representing a series of first PPG heart rate estimates, with wb-accelerometer peaks, representative of a series of second wb-accelerometer heart rate estimates. PPG peaks at frequencies that are similar to wb-accelerometer peaks have a higher probability of being true HR readings instead of MA. Conversely, PPG peaks at frequencies that are not substantially similar to wb-accelerometer peaks can be disregarded as related to MA or other noise. Comparing PPG HR estimates to wb-accelerometer HR estimates, including identifying differences and similarities between the two HR estimates, can improve MA rejection and result in a higher quality and a more accurate final HR estimate.

It is to be understood that although PPG module 100 is illustrated with a specific configuration, other arrangements of these components are within the scope of this disclosure. In other examples, module 100 can be included or arranged within a user device, such as an ear-worn device, such as, for example, an earbud, a mechanical watch, a smart watch, a smart ring, a cell phone, headphone, armband, or a laptop computer. In other examples, module 100 can be integrated into jewelry, such as a pendant, necklace, bangle, earring, armband, ring, anklet, or other jewelry.

FIG. 2 illustrates additional aspects of electronics 199. Although the description in FIG. 2 is given with respect to electronics 199, a person of skill in the art should understand that in some examples AFE 140 and electronics 199 can be combined or operate collectively. Illustrated in FIG. 2 is a bidirectional arrow indicating that communication between AFE 140 and electronics 199 can occur bidirectionally.

Electronics 199 may contain a power source 190, processor(s) 191, memory 192, data 193, a user interface 194, a display interface 195, communication interface(s) 197, and instructions 198, but need not include all such components depending on where electronics 199 is located. For example, electronics 199 can include a processing device, including a central processing unit. Furthermore, in some examples when the electronics 199 is positioned within an earbud, display interface 195 may not be included, but when electronics 199 is positioned within a smart watch, display interface 195 may be included. The power source may be any suitable power source to generate electricity, such as a battery, a chemical cell, a capacitor, a solar panel, or an inductive charger. Processor(s) 191 may be any conventional processors, such as commercially available microprocessors or application-specific integrated circuits (ASICs); memory, which may store information that is accessible by the processors including instructions that may be executed by the processors, and data. Memory 192 may be of a type of memory operative to store information accessible by the processors, including a non-transitory computer-readable medium, or other medium that stores data that may be read with the aid of an electronic device, such as a hard-drive, memory card, read-only memory (“ROM”), random access memory (“RAM”), optical disks, as well as other write-capable and read-only memories. The subject matter disclosed herein may include different combinations of the foregoing, whereby different portions of the instructions and data are stored on different types of media. Data 193 of electronics 199 may be retrieved, stored or modified by the processors in accordance with the instructions 198. For instance, although the present disclosure is not limited by a particular data structure, data 193 may be stored in computer registers, in a relational database as a table having a plurality of different fields and records, XML documents, or flat files. Data 193 may also be formatted in a computer-readable format such as, but not limited to, binary values, ASCII or Unicode. Moreover, data 193 may comprise information sufficient to identify the relevant information, such as numbers, descriptive text, proprietary codes, pointers, references to data stored in other memories (including other network locations) or information that is used by a function to calculate the relevant data.

Instructions 198 may control various components and functions of PPG module 100. For example, instructions 198 may be executed to selectively activate light source 110 or process information obtained by photodetector 120 or wb-accelerometer sensor 132. In some examples, algorithms can be included as a subset of or otherwise as part of instructions 198 included in electronics 199. Instructions 198 can include algorithms to interpret or process information received, such as information received through or generated by analyzing a ray received at a photodetector, PPG data signal 143, wb-accelerometer data signal 145, or information stored in memory. For example, physical parameters of the user can be extracted 16 or analyzed through algorithms. Without limitation the algorithms could use any or all information about the waveform, such as shape, frequency, or period of a wave, Fourier analysis of the signal, harmonic analysis, pulse width, pulse area, peak to peak interval, pulse interval, intensity or amount of light received by a photodetector, wavelength shift, or derivatives of the signal generated or received by photodetector 120 or wb-accelerometer data signal 145. Other algorithms can be included to calculate absorption of oxygen in oxyhemoglobin and deoxyhemoglobin, heart arrhythmias, heart rate, premature ventricular contractions, missed beats, systolic and diastolic peaks, and large artery stiffness index. In yet other examples, artificial learning or machine learning algorithms can be used in both deterministic and nondeterministic ways to extract information related to a physical condition of a user such as blood pressure and stress levels, from, for example, heart rate variability. In some examples, the algorithms can be modified or use information input by a user into memory of electronics 199 such as the user's weight, height, age, cholesterol, genetic information, body fat percentage, or other physical parameter. In other examples, machine learning algorithms can be used to detect and monitor for known or undetected health conditions, such as an arrhythmia, based on information generated by the photodetectors and/or processors. Instructions 198 can be stored in a memory 192 coupled to the processor(s) 191.

User interface 194 may be an optional screen which allows a user to interact with PPG module 100, such as a touch screen or buttons. Display 195 can be an LCD, LED, mobile phone display, electronic ink, or other display to display information about PPG module 100. User interface 194 can allow for both input from a user and output to a user. In some examples, the user interface 194 can be part of electronics 199 or PPG module 100, while in other examples, the user interface can be considered part of user device.

Communication interface(s) 197 can include hardware and software to enable communication of data over standards such as Wi-Fi, Bluetooth, infrared, radio-wave, and/or other analog and digital communication standards. Communication interface(s) 197 allow for electronics 199 to be updated and information generated by PPG module 100 to be shared with other devices. In some examples, communication interface(s) 197 can send historical information stored in memory 192 to another user device for display, storage, or further analysis. In other examples, communication interface(s) 197 can send the signal generated by the photodetector to another user device in real-time or afterwards for display on that device. In other examples, communication interface(s) 197 can communicate to another PPG module. Communication interface(s) 197 can include bluetooth, Wi-Fi, Gazelle, ANT, LTE, WCDMA, or other wireless protocols and hardware which enable communication between two devices.

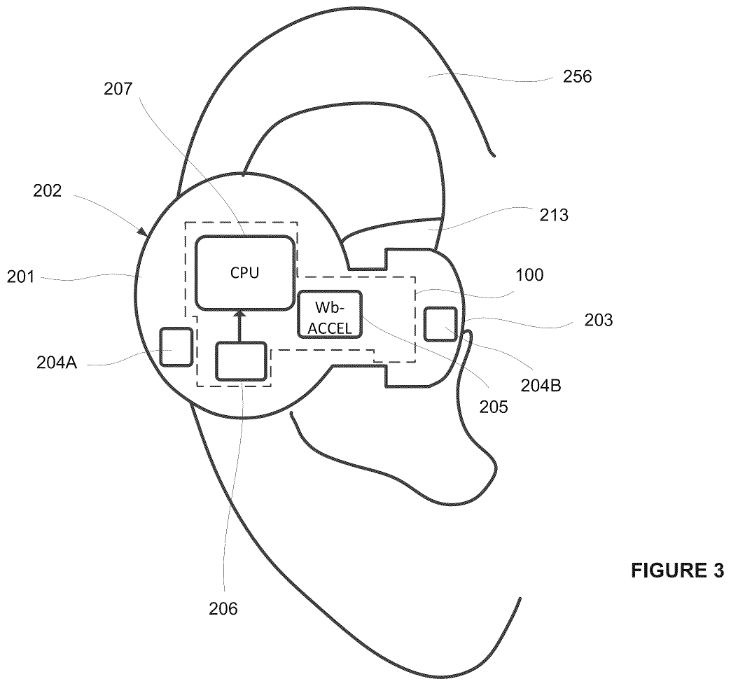

FIG. 3 illustrates a schematic view of an example user device 202. In this example, user device 202 may be an earbud sized to be worn in an ear 256 of a user. The user device can include a housing 201, and an earbud tip 203 which is intended to be positioned within the ear canal of a user and which may be formed by a portion of the housing 201. User device 202 can optionally include a feed forward microphone 204A and/or a feedback microphone 204B. User device 202 can contain PPG module 100 to perform PPG related functions, and can further include a wb-accelerometer sensor 205, PPG sensor 206, and a central processing unit (“CPU”) 207 including a processing device receiving data from the PPG sensor 206 and the wb-accelerometer sensor 205. In some examples, the feedback microphone 204B can be part of the PPG module and can be separately or additionally used to calculate heart rate estimates. The portion of housing 201 to be inserted into the ear 256 of a user, will contact the skin of user 213. When the user device 202 is positioned within the user's ear, a seal is typically formed between the ear of the user and the user device 202, such that the ear cavity is isolated from the outside world. Wb-accelerometer sensor 205 can sense user heart rate, and specifically vibrations in the ear propagating through tissue and bone as a result of user pulse or heart beat inside the ear canal. Additional components can be incorporated into user device 202 or in housing 201, as further described above with reference to FIGS. 1-3.

Wb-accelerometer sensor 205 may be a dedicated sensor that detects health-related characteristics of a user, including heart rate. Wb-accelerometer sensor 205 may also be a multi-purpose sensor that can additionally be used to improve or enhance the wb-accelerometer characteristics generated by the earbud. For example, wb-accelerometer sensor 205 may be an existing wb-accelerometer that is used to detect speech generated by a user's vocal cords and unvoiced speech generated without use of vocal cords through vibrations propagating through tissue and bone. Such a wb-accelerometer can act as an additional microphone whose data is fused with traditional microphone data for acoustic noise suppression.

A PPG algorithm can be used to determine a PPG-based HR estimate based on information obtained from an ear of the user, and particularly PPG data and wb-accelerometer data associated with heart rate of a user. The PPG algorithm, for example, is carried out by the processing device of CPU 207 (in particular according to the instructions stored in a memory of CPU 207). A final HR estimate can be determined using various PPG algorithms that rely on PPG data and wb-accelerometer data in raw form, or that use PPG data and wb-accelerometer data to individually calculate initial PPG heart rate estimates and wb-accelerometer heart rate estimates, as noted above.

Heart rate estimates can be compared in a PPG algorithm to improve the quality of a final HR estimate. PPG heart rate estimates obtained from the PPG sensor 206 in the user's ear 256 and wb-accelerometer heart rate estimates obtained from the wb-accelerometer sensor 205 in the user's ear 256 can be independently computed and then compared to one another. PPG heart rate estimates that are substantially similar to wb-accelerometer heart rate estimates, can be further identified as “real” heart rates rather than MA or noise. In some examples, if the two heart rate estimates fall within a threshold they can be considered substantially similar. PPG heart rate estimates that are substantially different than the wb-accelerometer heart rate estimates, and for example, fall outside of a threshold, can then be identified as MA or other noise and disregarded. Identifying the PPG and wb-accelerometer heart rate estimates that are substantially similar, and those that are substantially different, can improve the quality and accuracy of the final HR estimates. A set range of what is substantially similar can be pre-determined ahead of time. For example, substantial similarity can be designated as those heart rate estimates where the PPG heart rate does not differ by more than five heartbeats per minute from a wb-accelerometer heart rate estimate. The PPG heart rate estimate will be considered more likely to be a true heart rate when it does not differ by more than five heartbeats per minute from an wb-accelerometer heart rate estimate. Those PPG heart rate estimates that differ by more than five heartbeats per minute can be disregarded or subtracted out as related to MA or other noise. In other examples, the threshold may be less than or more than five heartbeats per minute.

PPG data and web-accelerometer rate data, or PPG heart rate estimates and wb-accelerometer heart rate estimates, can be combined additively in a PPG algorithm to obtain a final HR estimate. In one example, the final HR estimate can be based on a combination of PPG data detected by the PPG sensor and wb-accelerometer data detected by the wb-accelerometer sensor of the user device 202. That is, final HR estimates are made by combining data together, to calculate heart rate estimates using fusion techniques.

In another example, the final HR estimate can be based on a combination of a first PPG heart rate estimate and an independently-calculated second wb-accelerometer heart rate estimate. Determining a final PPG HR estimate by combining wb-accelerometer data and PPG data, or alternatively, wb-accelerometer heart rate estimates in PPG heart rate algorithms can improve a user's overall heart rate estimates.

Further, although user device 202 is shown as including a CPU 207, the PPG algorithms can be incorporated within another computing device, such as a smart watch, smart phone, desktop computer and the like. For example, as will be discussed in more detail below, information or data from the user device 202 can be transmitted to a processor of another device, such as a mobile device or a smart watch.

Although an earbud is illustrated as user device 202, a person of skill in the art will appreciate that user device 202 can take on a variety of forms. User device 202 can be, for example, a smartwatch, a health sensor, or other wearable electronics that are capable of detecting heart rate, such as through use of a PPG sensor and/or a wb-accelerometer sensor, including a ring, necklace, or other piece of jewelry that can be positioned to detect heart rate characteristics of a user.

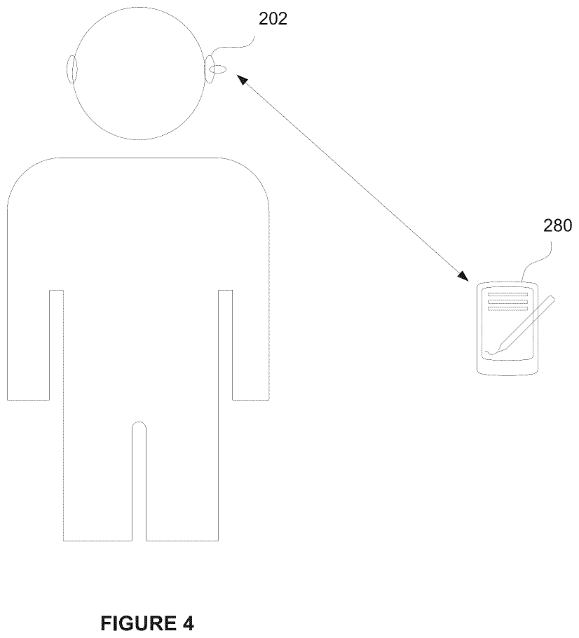

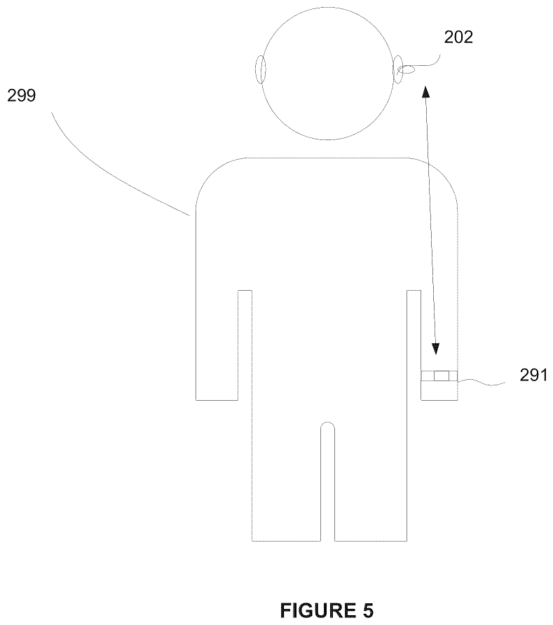

FIG. 4 illustrates communication between two user devices, a first user device 202 and a second user device 280, and which can display characteristics about the user received from user device 202. In one example, where user device 202 is an earbud, the secondary user device 280 may be a smart phone that may or may not include PPG module, such as PPG module 100. The final HR estimate can be calculated at the first user device 202 and then transmitted to user device 280 for display. The final HR estimate can alternatively be calculated by user device 202, based on information received from user device 202. To calculate a final HR estimate, user device 202 can receive PPG data associated with heart rate and wb-accelerometer data associated with heart rate from user device 202, and then calculate a final HR estimate based upon a combination of the PPG data and wb-accelerometer data. Alternatively, user device 280 can receive either or both a pre-calculated PPG heart rate estimate and a pre-calculated wb-accelerometer heart rate estimate from user device 202 and combine the estimates to determine the final HR estimate. Other example secondary devices can be alternatively used in connection with user device 202, such as a watch, pendant, computer or other device. FIG. 5 illustrates an example system in which user device 280 communicates with a smart watch 291. Although the same reference numerals are used for the devices in FIGS. 4-5, as the devices in FIG. 3, these devices need not be the same devices.

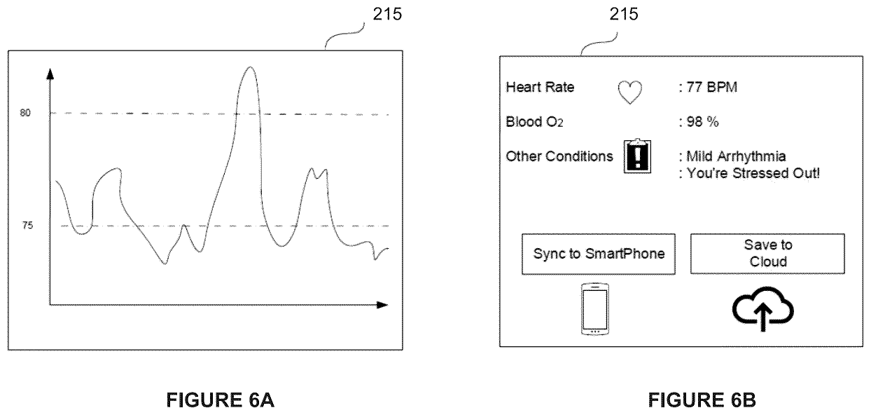

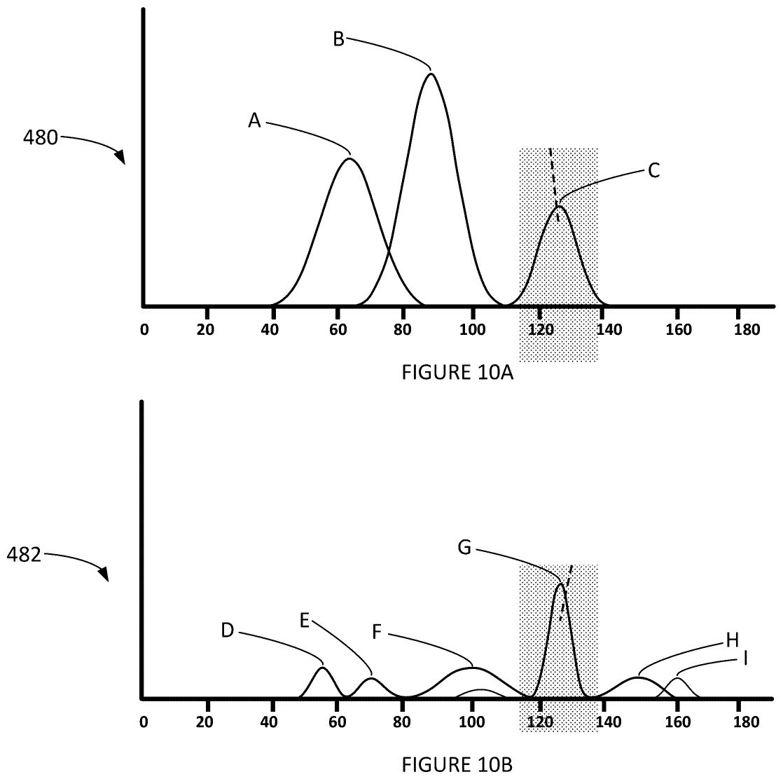

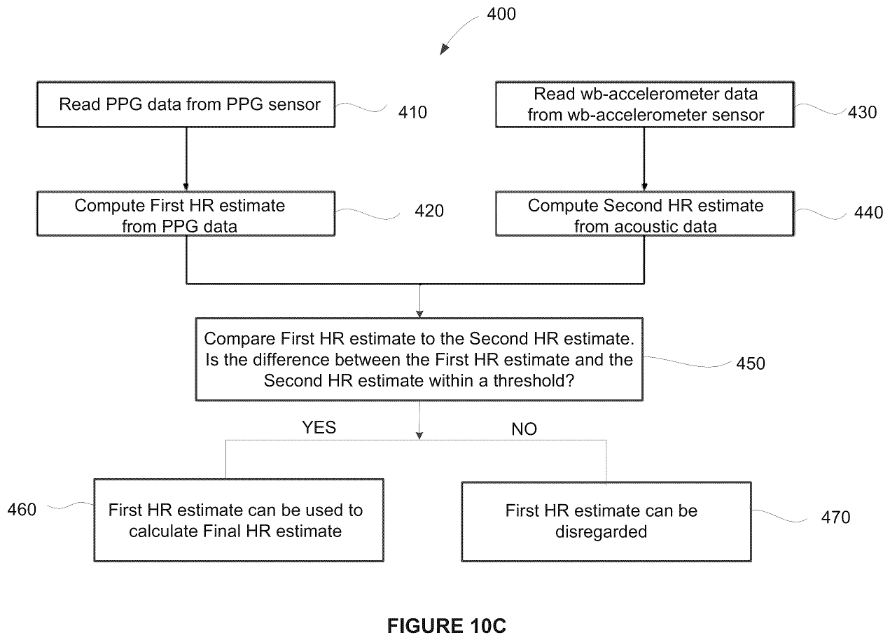

FIG. 6A and FIG. 6B illustrate example formats for displaying information about a physical condition of a user on a display 215 of a user device, which can be a smart phone, desktop computer, watch, and the like. Display 215 can be similar to display 195 described above. FIG. 6A illustrates a graph of the heart rate of a user of a device, such as device 200. This graphical view can be updated in real time to display a trailing number of seconds of the heartbeat of the user. In some examples, in “real time” or “real-time” can mean the execution of data instructions, or algorithms in a short time period, which can provide near-instantaneous output to a user or user device. The heart rate being displayed can be obtained from the methods described below with reference to FIGS. 10A-12. FIG. 6B illustrates displaying information about a physical condition of a user in a textual format. For example, FIG. 6B illustrates the current heart rate in beats-per-minute (BPM), the current blood oxygen saturation level, and any other conditions that may be of value to the user, such as an arrhythmia. Although the examples given are for cardiovascular conditions, other aspects of the heart can be monitored. FIG. 6B also illustrates other options, such as the ability to sync the information to another user device, such as a smartphone, or saving the information to another storage unit, such as the internet or to the cloud. Although information is displayed in a visual format, in other examples, the information may be provided through an auditory method. Information being displayed in FIG. 6B can be derived from the methods described below with respect to FIGS. 10A-12.

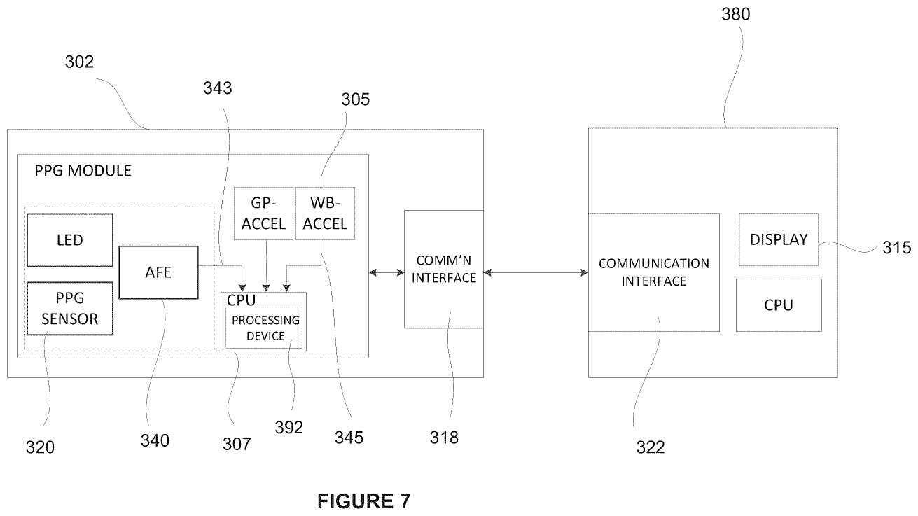

FIG. 7 is a schematic illustrating communication between two user devices in a system: user device 302, which can be configured as explained above, and user device 380. In this example, user device 302 may be an earbud and user device 380 can be a smart phone, but other combinations of user devices can also be implemented. As shown, a processing device 392 can be part of CPU 307 within user device 302. For example, processing device 392 can include any portion of CPU 307 necessary to perform processing functions, including functions related to processing PPG and wb-accelerometer data, and computing the PPG algorithm. Processing device 392, for example, may comprise a given portion of logic circuitry within CPU 307 that operates in accordance with instructions implementing algorithms, processes or methods discussed herein. The given portion may include as much processing capability needed to implement the processes, etc., and depend on the complexity of the algorithm.

In this example, PPG algorithms, and any related algorithms, may be calculated within user device 302. For example, a final HR estimate may be based on first combining PPG data 343 received from the PPG sensor 320 via AFE 340 and wb-accelerometer data 345 received from the wb-accelerometer sensor 305, and then calculating the final HR estimate based on the combined data. In another example, the CPU 307 may receive PPG heart rate data from AFE 340 and the processing device 392 of the CPU may calculate a first PPG heart rate estimate, the CPU 307 may then receive wb-accelerometer heart rate data and calculate a second heart rate estimate. The calculated first PPG heart rate estimate and the second wb-accelerometer heart rate estimate may then be combined to obtain a final HR estimate. Once the final HR estimate is determined by the PPG processing device 392, the final HR estimate may then be displayed to a user on a display of a user device. Finally, in another example, the PPG heart rate estimate and wb-accelerometer heart rate estimates can be compared to identify heart rate estimates that are substantially similar and/or substantially different, as discussed above.

When user device 302 is an earbud, which may not include a display device, the final HR estimate may be communicated via communication interface 318 to another device having a display, such as user device 380. Communication interface 322 of device 380 may receive the final HR estimate which can be provided on display 315 of device 380. In other examples, the user device display may be associated with a computer, or other electronic device capable of receiving information from user device 302.

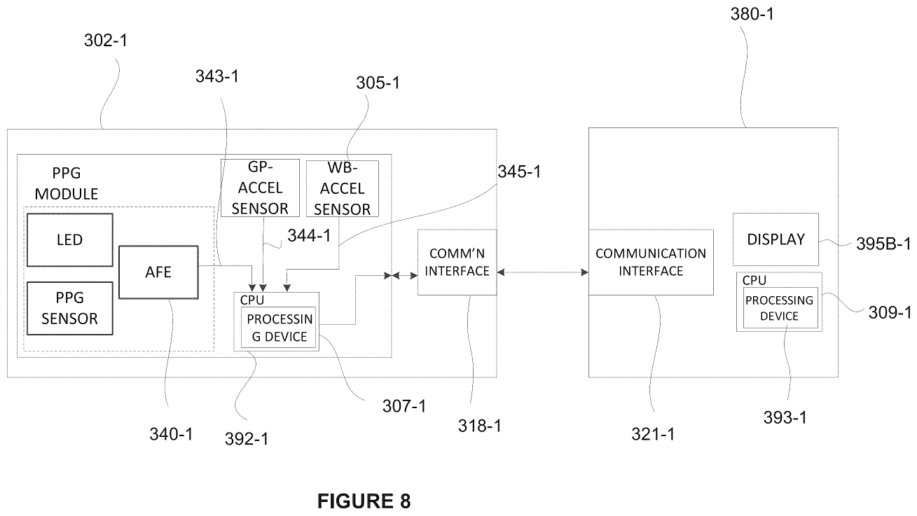

FIG. 8 is a schematic illustrating another example system showing communication between two user devices, first user device 302-1 and second user device 380-1, both of which can be configured as explained above. In this example, a processing device 393-1 can be part of the second user device 380-1, such that some or all of the PPG algorithms are provided or calculated within user device 380-1.

For example, PPG data 343-1 and wb-accelerometer data 345-1 may be communicated via communication interface 318-1 of first user device to the CPU 393-1 of the second user device 380-1 via communication interface 321-1. Processing device 393-1 of the second user device 380-1 may first combine the PPG data 343-1 and the wb-accelerometer data 345-1, and then calculate the final HR estimate using the combination of data in a PPG algorithm. Alternatively, the second user device 380-1 may use the PPG data to first calculate a PPG heart rate estimate and the wb-accelerometer data to calculate a wb-accelerometer heart rate estimate. The PPG heart rate estimate and the wb-accelerometer heart rate estimate may then be combined to determine a final heart rate estimate.

In another example, the CPU 307-1 may receive PPG data 343-1 from AFE 340-1 and the processing device 392-1 may calculate a first PPG heart rate estimate. The CPU 307-1 may then receive wb-accelerometer heart rate data 345-1 received from wb-accelerometer sensor 305-1 of user device 302-1 and calculate a second wb-accelerometer heart rate estimate. The first and second heart rate estimates may then be relayed to the second user device 380-1 via the respective communication interfaces 318-1 and 321-1. The processing device 393-1 of the second user device 380-1 can then combine the first and second heart rate estimates in a PPG algorithm to calculate a final HR estimate. The PPG based heart rate estimate may then be displayed to a user on display interface 395B-1 of user device 380-1, or alternatively, on a display interface (not shown) of user device 302-1.

In another example, PPG heart rate estimates and wb-accelerometer heart rate estimates can be identified by the processing device 392-1 of first user device 302-1. Processing device 392-1 may process the PPG data and wb-accelerometer data in the time or frequency domain to identify maximum peaks in the PPG data and wb-accelerometer data as representative of heart rate estimates. Either one or both of the PPG heart rate estimates and wb-accelerometer heart rate estimates can be relayed to the CPU 309-1 of second user device 380-1 via communication interface 318-1 and communication interface 321-1. The processing device 393-1 can then compare the two heart rate estimates to identify heart rate estimates that are substantially similar, as discussed above, as well as those that may be disregarded as related to MA.

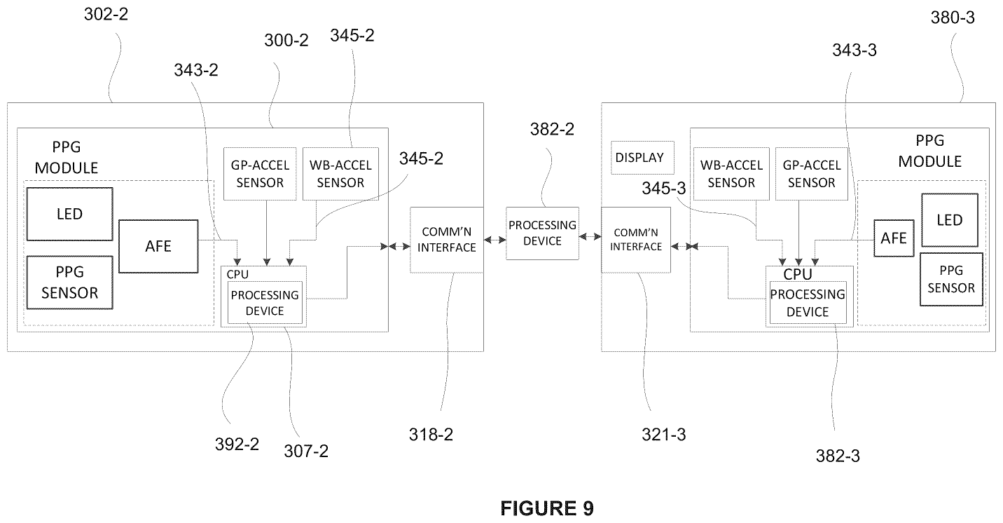

FIG. 9 is a schematic illustrating another example system showing two user devices, first user device 302-2 and second user device 380-3, and a processing device 382-2. In one example, first user device 302-2 may be earbuds, the second user device 380-3 may be a watch, and processing device 382-2 can be part of another device, such as a laptop, desktop computer, or other device capable of receiving wireless communication from the respective communication interfaces 318-2, 321-3 of respective user device 302-2 and user device 380-3. As shown, PPG data 343-2 and wb-accelerometer data 345-2 associated with heart rate can be communicated to an external processing device 382-2 via communication device 318-2. Processing device 382-2 can combine the PPG data 343-2 and wb-accelerometer data 345-2, and then calculate the final HR estimate. This information can be further communicated to second user device 380-3 for display to the user.

In another example, processing device 392-2 of CPU 307-2 of PPG module 300-2 of first user device 302-2 can calculate a PPG heart rate estimate and a wb-accelerometer heart rate estimate, and then dispatch the information to an external processing device 382-2 via communication interface 318-2. Processing device 382-2 can then combine the PPG heart rate estimate and the wb-accelerometer heart rate estimate to determine the final HR estimate. In the example where user device 302-2 does not include a display, processing device 382-2 can communicate the final HR estimate to user device 380-3. Alternatively, if user device 302-2 includes a display, processing device may either communicate the final HR estimate back to user device 302-2 or display the information on the second user device 380-3.

In another example, PPG heart rate estimates and wb-accelerometer heart rate estimates can be computed by the processing device 382-2. In a time or frequency domain of PPG data and wb-accelerometer data, PPG and wb-accelerometer data collected by either or both of the first or second user devices 302-2 and 380-3 can be relayed to processing device 382-2 via the respective communication interfaces 318-2 or 321-3. Processing device 382-2 can identify maximum peaks in the PPG data and wb-accelerometer data as representative of heart rate estimates. The processing device 382-2 can then compare the two heart rate estimates to identify heart rate estimates that are substantially similar, as discussed above, as well as those that may be disregarded as related to MA.

PPG and wb-accelerometer data may be collected by either the first or second user devices 302-2 and 380-3 and relayed to processing device 382-2 via the respective communication interfaces 318-2 or 321-3. In one example, the first user device 302-2 may be a device, such as an earbud, that communicates wb-accelerometer data 345-2 to the processing device 382-2 and the second user device 380-3 may be, for example, a watch that communicates PPG data 343-3 to the processing device 382-2. Processing device 382-2 can further process wb-accelerometer data 345-2 received from the first user device 302-2 and the PPG data 343-3 received from second user device 380-3 according to any of the methods described above to compute one or more heart rate estimates.

Further, the transfer of a first heart rate estimate signal and second heart rate estimate signal, or PPG data and wb-accelerometer data, can include not only a single piece of information being transferred between the device, but rather a continuous or near-continuous signal or data being transmitted which includes time-series or other information. In other examples, other processed signals can be transmitted, such as derivative metrics which are derived from the first heart rate estimate signal or the second heart rate estimate signal, or PPG data signal and wb-accelerometer data signal respectively.

### EXAMPLE METHODS

Example methods of providing a final PPG based heart rate estimate based on information obtained from a wb-accelerometer sensor and a PPG sensor are disclosed. In a first example, in a frequency domain, wb-accelerometer heart rate estimate peak magnitudes are compared to PPG heart rate estimate peak magnitudes to disambiguate peaks related to MA. Identical or substantially identical peaks between the wb-accelerometer heart rate estimate and the PPG heart rate estimate are identified as the “true” HR estimate used in the final HR estimate. Non-identical peaks in differing frequencies are disregarded as related to MA or other noise and not representative of the user's heart rate estimate. In a second example, PPG data and wb-accelerometer data are additively combined to create a higher quality PPG signal, which is then processed by a PPG algorithm to determine a final HR estimate. In a third example, the wb-accelerometer data is used to estimate a first wb-accelerometer heart rate independently of the PPG data. A second heart rate estimate is determined based on PPG data, and the first heart rate estimate and the second heart rate estimates are combined to generate a final HR estimate. Other methods of calculating a final HR estimate based upon wb-accelerometer data and PPG data may also be implemented. All of these methods can be used to improve the accuracy of PPG systems.

### First Example Method

Known PPG heart rate algorithms may attempt to filter out artifacts related to motion using general purpose accelerometer data, but peaks associated with MA still remain. Instead of determining final HR estimates based on PPG heart rate estimates alone (or optionally PPG heart rate estimates filtered with gp-accelerometer data), accelerometer heart rate estimates can be compared with preliminary PPG heart rate estimates to identify final HR estimates.

As shown in FIG. 10A, example PPG data 480 is shown in a frequency domain graph and can be used to determine heart rate estimates in beats per minute. The x-axis illustrates beats per minute and the y-axis represents PPG heart rate peak magnitude. An initial PPG algorithm can be used to calculate and identify maximum peak values, such as from Peak A, Peak B, and Peak C. The maximum peak values represent initial heart rate estimates based on PPG data detected by a PPG sensor. For example, Peak A may be 65 bpm, Peak B may be 90 bpm, and Peak C may be 125 bpm.

As shown in FIG. 10B, example wb-accelerometer data 482 is shown in frequency domain graph and can also be used to determine heart rate estimates in beats per minute. Wb-accelerometer algorithms can be used to calculate and identify peak values, such as Peaks D-I, which are representative of heart rate estimates based on wb-accelerometer data detected by a wb-accelerometer sensor. For example, Peak D indicates a heart rate estimate of 55 bpm, Peak E may be 65 bpm, Peak F may be 100 bpm, Peak G may be 125 bpm, Peak H may be 150 bpm, and Peak I may have a heart rate estimate of 160 bpm.

The magnitude peaks of the PPG heart rate estimate may then be compared to the peaks of the wb-accelerometer heart rate estimates to identify frequencies where peaks are found in both data sets. A comparison of wb-accelerometer heart rate estimates and PPG heart rate estimates, represented by peaks, reflects that both the PPG heart rate estimate and wb-accelerometer heart rate estimate have a peak of 125 bpm. This is a good indicator and adds confidence that 125 bpm is the true heart rate estimate and that the remaining peaks can be disregarded as related to MA or other noise. Thus, 125 bpm can be used to compute the true or final HR estimate.

In some examples, a threshold for determining whether peaks are substantially at the same frequency can be predetermined. The threshold can represent the maximum difference between the PPG heart rate estimate and the wb-accelerometer heart rate estimate that can qualify the PPG heart rate estimate as being the true HR estimate. For example, the threshold or maximum difference may be 5 bpm, such that any PPG heart rate estimate that differs from the wb-accelerometer heart rate estimate by more than 5 bpm should be disregarded as related to a motion artifact. In other examples, the maximum difference between the wb-accelerometer heart rate estimate and PPG heart rate estimates may be less than 5 bpm or greater than 5 bpm. In other examples, the threshold is dynamic and changes over time.

Referring to 10C, an example flow chart illustrates an example method 400 of calculating a final HR estimate based on wb-accelerometer and PPG heart rate estimates. At block 410, PPG data can be read from a PPG sensor. As explained above with reference to FIG. 1A, a PPG sensor, such as a photodetector or photodiode, can be embedded into a PPG module of a user device.

At block 420, a first PPG heart rate estimate can be computed based on the received PPG data. In particular, the PPG heart rate estimates are based on identification of peaks in the PPG data. The calculation of the first PPG heart rate can occur locally at the user device or it can be calculated at another device. For example, the PPG heart rate estimate can be calculated at electronics 199 of PPG module 100, which can be embedded into the user device.

At block 430, wb-accelerometer data can be read from a wb-accelerometer sensor. As explained above, a wb-accelerometer sensor can be embedded into the PPG module. Wb-accelerometer sensor 132 may be configured to sense user heart rate, such as wb-accelerometer waves reflecting movement caused by the user pulse or heartbeat.

At block 440, a second heart rate estimate can be computed from the wb-accelerometer data. In particular, the wb-accelerometer heart rate estimates are based on identification of peaks in the wb-accelerometer data. The computation of PPG and wb-accelerometer based heart rate at blocks 420 and 440 can occur simultaneously, close in time to one another, or in “real-time” with one another.

At block 450, the first heart rate estimate and the second heart rate estimate can be compared to one another to determine if the first heart rate estimate is the same or substantially the same as the second heart rate estimate. In some examples, a determination can be made whether any differences between the first and second heart rate estimates fall within a threshold.

If yes, at block 460, the first heart rate estimate is used to compute the final HR estimate. Further algorithms can be utilized to achieve a more accurate number.

If no, at block 470, the first HR estimates can be disregarded as motion artifacts or other noise.

### Second Example Method

In a second example method, a PPG data signal and a wb-accelerometer signal received from the user device may be combined with a wb-accelerometer data signal to produce a PPG-based or final HR estimate. Such method is more accurate and less susceptible to motion artifacts than reliance on PPG data alone or traditional methods of PPG calculation.

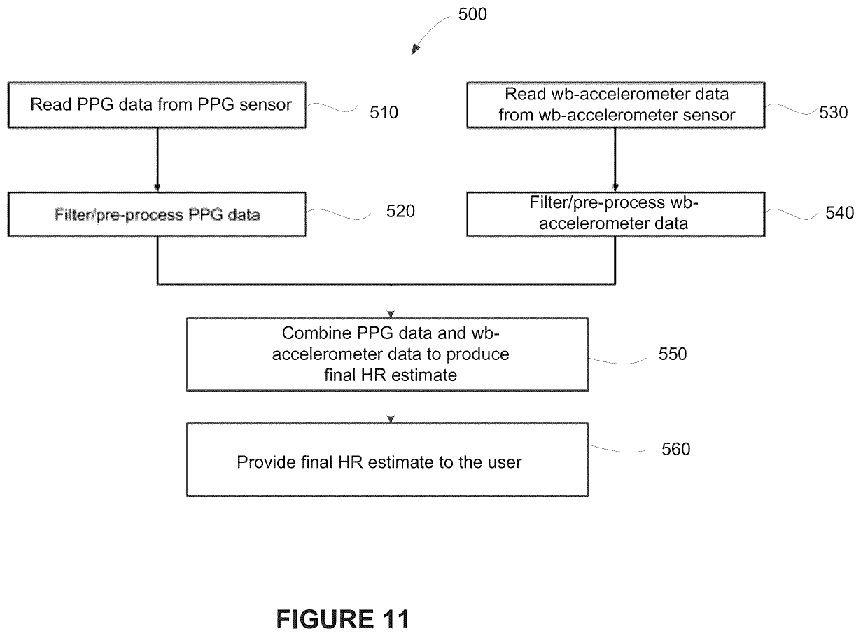

FIG. 11 illustrates an example method 500. At block 510, PPG data from a user device can be read from a PPG sensor. As explained above with reference to FIG. 1A, a PPG sensor can be embedded into a PPG module of a user device and obtain data from a photodetector/photodiode. As discussed above, PPG data associated with heart rate and received from a photodetector can be converted to digital samples.

At block 520, the PPG data can be filtered or pre-processed to remove noise outside of the area of interest. For example, additional data such as gp-accelerometer data can be used to filter PPG data.

At block 530, data can be read from a wb-accelerometer sensor. As explained above, a wb-accelerometer sensor 132 can be embedded into the PPG module 100 of a user device. Wb-accelerometer sensor 132 may be configured to sense user heart rate, and specifically wb-accelerometer waves reflecting air pressure changes caused by the user pulse or heartbeat.

At block 540, the wb-accelerometer data may be filtered. For example, wb-accelerometer data would be pre-processed to filter out noise and other signals out of the normal band of heart rate frequencies. The wb-accelerometer data may also need to be scaled or normalized prior to combination with the PPG data.

It is to be appreciated that the actions at block 510 and 520 can occur simultaneously with the actions at block 530 and 540, i.e., close in time, or in ‘real-time” with one another.

At block 550, the final HR estimate can be calculated based on the filtered PPG data and the filtered wb-accelerometer data. For example, the PPG and wb-accelerometer data may be combined using a weighted combination. Samples from each signal may be scaled with a weighting factor and summed, such as in the example PPG algorithm or formula below:

signal_{final}(n)=ω1(n)·signal_{ppg}(n)+ω₂(n)·signal_{wb-accelerometer}(n)

where:
(n) is the sample index which represents a point in time
ω₁(n) is a weight assigned to the PPG data
signal_{ppg}(n) is the signal derived from the PPG sensor
ω₂(n) is a weight assigned to the wb-accelerometer data
signal_{wb-accelerometer}(n) is the signal derived from the wb-accelerometer sensor
   The weights can be static. In other examples, the weights can be based on historically accurate information. In other examples, the weights can be changing dynamically. Using the combined PPG data and wb-accelerometer data in the PPG algorithm, can help to improve accuracy of the final HR estimate.

Alternative methods and algorithms for combining the PPG data and wb-accelerometer data can be implemented. For example, simple averaging, weighted averaging, or more sophisticated combinations such as kalman filters and bayesian networks may be utilized. In cases where the PPG algorithm implementation is based on AI techniques, the signals can be combined using convolutional neural networks or similar machine learning techniques for heart beat pattern recognition.

At block 560, the final HR estimate can optionally be provided to the user. In some examples, the final HR estimate can be provided to a display of the same or different user device. The final HR estimate can also be provided using auditory methods, such as through beeps or by using text-to-speech to provide the final HR estimate to the user through synthesized speech.

### Third Example Method

In a third example method, a first PPG heart rate estimate and a second wb-accelerometer heart rate estimate are combined together to determine a final HR estimate. A first heart rate estimate may be computed based on data derived from PPG data derived from a PPG sensor. A second heart rate estimate may be independently computed based on wb-accelerometer data derived from a wb-accelerometer sensor. Once the first and second heart rates are independently calculated, they may be combined together in a PPG algorithm to obtain a third and final HR estimate. The final HR estimate on average can be more accurate and less susceptible errors which can be easily introduced into the final HR estimates.

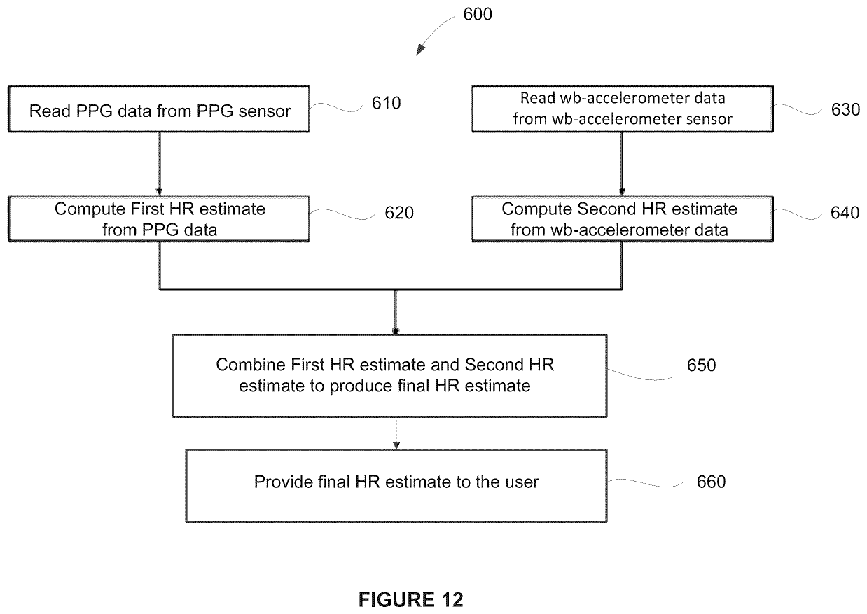

Referring to FIG. 12, another example method 600 is shown. At block 610, PPG data can be read from a PPG sensor. As explained above with reference to FIG. 1A, a PPG sensor can be embedded into a PPG module of a user device and obtain data from a photodetector/photodiode.

At block 620, a first PPG heart rate estimate can be computed based on the received PPG data. The calculation of the first PPG heart rate can occur locally at the user device or it can be calculated at another device. For example, the PPG heart rate estimate can be calculated at electronics 199 of PPG module 100, which can be embedded into the user device.

At block 630, wb-accelerometer data can be read from a wb-accelerometer sensor. As explained above, a wb-accelerometer sensor can be embedded into the PPG module of a user device. In some examples, wb-accelerometer sensor 132 may be configured to sense user heart rate, and specifically wb-accelerometer vibrations reflecting air pressure changes caused by the user pulse or heart beat.

At block 640, a second heart rate estimate can be computed from the wb-accelerometer data. The computation of PPG and wb-accelerometer based heart rate at blocks 520 and 540 can occur simultaneously, close in time to one another, or in “real-time” with one another.

At block 650, the first heart rate estimate and the second heart rate estimate can be combined together to produce the final HR estimate. The first and second heart rate estimates can be combined by various fusion techniques. As in the previous example, one technique is to use a weighted combination. An example formula for a weighted combination is shown below:

signal_{final}(n)=ω1(n)·signal_{ppg}(n)+ω₂(n)·signal_{wb-accelerometer}(n)

where:
ω₁(n) is a weight assigned to the first heart rate estimate derived from PPG data
signal_{ppg}(n) is the signal derived from the PPG sensor
ω₂(n) is a weight assigned to the second heart rate estimate derived from wb-accelerometer data
signal_{wb-accelerometer}(n) is the signal derived from the wb-accelerometer data

In some examples, the weights can be static. In other examples, the weights can be based on historically accurate information. In other examples, the weights can be changing dynamically.

At block 660, the final HR estimate can optionally be provided to the user. The final HR estimate can be displayed to the user on a display of the user device. The user device may be the user device containing the PPG and wb-accelerometer sensors, or another user device altogether. The final HR estimate can alternatively or additionally be provided in other auditory methods, such as through beeps or by using text-to-speech to provide the final HR estimate to the user through synthesized speech. In one example, where the user device is an earbud, results can be audibly provided over the earbud. Further, when the final HR is at a rate that may be indicative of a health problem, alarms may be triggered to notify the user of the condition.

While this disclosure contains many specific implementation details, these should not be construed as limitations on the scope of what may be claimed, but rather as descriptions of features specific to particular implementations. Certain features that are described in this specification in the context of separate implementations may also be implemented in combination in a single implementation. Conversely, various features that are described in the context of a single implementation may also be implemented in multiple implementations separately or in any suitable sub-combination. Moreover, although features may be described above as acting in certain combinations and even initially claimed as such, one or more features from a claimed combination may in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous.

References to “or” may be construed as inclusive so that any terms described using “or” may indicate any of a single, more than one, and all of the described terms. The labels “first,” “second,” “third,” and so forth are not necessarily meant to indicate an ordering and are generally used merely to distinguish between like or similar items or elements.

Various modifications to the implementations described in this disclosure may be readily apparent to those skilled in the art, and the generic principles defined herein may be applied to other implementations without departing from the spirit or scope of this disclosure. Thus, the claims are not intended to be limited to the implementations shown herein, but are to be accorded the widest scope consistent with this disclosure, the principles and the novel features disclosed herein.

1. An apparatus, comprising:
a processing device coupled to a memory storing instructions, the instructions causing the processing device to:
receive photoplethysmography (PPG) data derived from signals associated with at least one PPG sensor;
receive wideband accelerometer data derived from signals associated with at least one wideband accelerometer sensor configured to sense a heart rate of a human subject; and
combine the PPG data and the wideband accelerometer data to generate a heart rate estimate.

2. The apparatus of claim 1, further comprising at least one of the at least one PPG sensor and the at least one wideband accelerometer sensor.

3. The apparatus of claim 2, wherein the apparatus further comprises both the at least one PPG sensor and the at least one wideband accelerometer sensor.

4. The apparatus of claim 1, wherein the instructions further cause the processing device to filter PPG data with general purpose accelerometer data from at least one general purpose accelerometer sensor.

5. The apparatus of claim 1, wherein the apparatus further comprises an ear-worn wearable device.

6. The apparatus of claim 1, wherein the apparatus further comprises a wrist worn device.

7. The apparatus of claim 1, further comprising at least one filter, the filter configured to filter signals outside a frequency band associated with heart rate frequencies from the wideband accelerometer data.

8. The apparatus of claim 7, wherein the frequency band comprises a band between 0.6 to 2 Hz.

9. The apparatus of claim 1, wherein the instructions further cause the processing device to normalize the wideband accelerometer data prior to combining the wideband accelerometer data with the PPG data.

10. The apparatus of claim 1, wherein the instructions further cause the processing device to combine using a weighted combination in which the PPG data and the wideband accelerometer data are scaled with a weighting factor and summed.

11. The apparatus of claim 1, wherein the instructions further cause the processing device to combine the PPG data and the wideband accelerometer data using one of simple averaging, weighted averaging, kalman filtering and bayesian networks.

12. The apparatus of claim 1, wherein the instructions further cause the processing device to identify noise artifacts in the PPG data based on the wideband accelerometer data.

13. The apparatus of claim 1, wherein the instructions further cause the processing device to combine one or more samples of wideband accelerometer data with one or more samples of PPG data by using the wide band accelerometer data as an additive source with the PPG data.

14. The apparatus of claim 1, wherein the instructions further cause the processing device to compute a confidence metric for a PPG heart rate estimate derived from the PPG data based at least on the wideband accelerometer data.

15. The apparatus of claim 1, wherein the processing device receives the PPG data and the wideband accelerometer data over a wireless link.

16. The apparatus of claim 1, wherein the generating of the heart rate estimate is based on:
a first heart rate estimate computed, by the processing device, from the PPG data; and
a second heart rate estimate computed, by the processing device, from the wideband accelerometer data.

17. (canceled)

18. The apparatus of claim 1, wherein the at least one wideband accelerometer sensor is designed to have a frequency response bandwidth of at least 2 kHz.

19. The apparatus of claim 1, where the instructions further cause the processing device to filter PPG data with general purpose accelerometer data derived from the at least one general purpose accelerometer sensor, and wherein the at least one general purpose accelerometer sensor detects frequency bandwidths less than 2 kHz.

20-32. (canceled)

33. A computer-implemented method of determining a heart rate estimate of a human subject, the method comprising:
receiving photoplethysmography (PPG) data derived from signals associated with at least one PPG sensor;
receiving, from at least one wideband accelerometer sensor, wideband accelerometer data derived from signals associated with a heart rate of the human subject; and
determining, at a processing device, the heart rate estimate of the human subject by combining the wideband accelerometer data and the PPG data.

34-39. (canceled)

40. The computer-implemented method of claim 33, wherein combining comprises summing a weighted combination of the wideband accelerometer data and the PPG data, wherein the PPG data and the wideband accelerometer data are scaled with a weighting factor.

41-59. (canceled)
